(19) United States
(12) Patent Application Publication
(10) Pub. No.: US 2021/0207186 A1
(43) Pub. Date: Jul. 8, 2021

(54) Method of Making Polynucleotides Using an Anion Toroidal Vortex

(71) Applicant: President and Fellows of Harvard College, Cambridge, MA (US)

(72) Inventors: George M. Church, Brookline, MA (US); Howon Lee, Allston, MA (US); Mirkó Palla, Newton, MA (US)

(21) Appl. No.: 15/999,618

(22) PCT Filed: Feb. 15, 2017
(86) PCT No.: PCT/US17/17918

§ 371 Date: Aug. 20, 2018

Related U.S. Application Data
(60) Provisional application No. 62/296,833, filed on Feb. 18, 2016.

(51) Int. Cl. C12P 19/34

(52) U.S. Cl. CPC C12P 19/34

(57) ABSTRACT

A method for making a polynucleotide is provided that includes (a) delivering one or more reaction reagents including an error prone or template independent DNA polymerase, cations and a selected nucleotide to a reaction site including an initiator sequence having a terminal nucleotide for a time period and under conditions sufficient to covalently add a desired number of the selected nucleotide to the terminal nucleotide at the 3′ end of the initiator such that the selected nucleotide becomes a terminal nucleotide, moving cations away from the initiator sequence using an anion toroidal vortex to inhibit covalent addition of the selected nucleotide by the error prone or template independent DNA polymerase, removing the reaction reagents from the reaction site, and (b) repeating step (a) until the polynucleotide is formed.

### RELATED APPLICATION DATA

This application claims priority to U.S. Provisional Application No. 62/296,833 filed on Feb. 18, 2016 which is hereby incorporated herein by reference in its entirety for all purposes.

### FIELD

The present invention relates in general to methods of making oligonucleotides and polynucleotides using enzymatic synthesis.

### BACKGROUND

Methods of making polynucleotides are known.

### SUMMARY

The disclosure provides methods of making a polynucleotide using one or more or a plurality of optically addressable virtual electrodes and an error prone or template independent DNA polymerase, cations and a selected nucleotide. An exemplary template independent DNA polymerase is terminal deoxynucleotidyl transferase (TdT) which is used to synthesize single strand DNA by incorporation of random nucleotides at the end of 3′ end of a initiator strand or growing oligonucleotide or polynucleotide. The disclosure provides use of a photoconductive amorphous silicon layer at a reaction site which becomes a virtual electrode and exerts electroosmotic force. Suitable photoconductive amorphous silicon layers can be fabricated by those of skill in the art. An exemplary fabrication facility is the Center for Nanoscale Systems. A commercially available light module can be used to illuminate the photoconductive amorphous silicon layer.

The disclosure provides a method for making a polynucleotide including (a) delivering one or more reaction reagents including an error prone or template independent DNA polymerase, cations and a selected nucleotide to a reaction site including an initiator sequence having a terminal nucleotide for a time period and under conditions sufficient to covalently add a desired number of the selected nucleotide to the terminal nucleotide at the 3′ end of the initiator such that the selected nucleotide becomes a terminal nucleotide, moving cations away from the initiator sequence using an anion toroidal vortex to inhibit covalent addition of the selected nucleotide by the error prone or template independent DNA polymerase, removing the reaction reagents from the reaction site, and (b) repeating step (a) until the polynucleotide is formed. The disclosure provides the anion toroidal vortex deactivates the error prone or template independent DNA polymerase. The disclosure provides the anion toroidal vortex localizes the cations away from the initiator sequence. The disclosure provides the anion toroidal vortex controls activity of the error prone or template independent DNA polymerase at the reaction site. The disclosure provides a single selected nucleotide is covalently added. The disclosure provides that one, two, three or four selected nucleotides are covalently added. The disclosure provides a plurality of selected nucleotides are covalently added. The disclosure provides the error prone template independent DNA polymerase is terminal deoxynucleotide transferase. The disclosure provides the anion toroidal vortex is created by an optically addressable virtual electrode. The disclosure provides a plurality of reaction sites where step (a) is performed. The disclosure provides the reaction site includes an amorphous silicon layer where electric impedance changes in response to light intensity and a tangential electric field is generated. The disclosure provides an anion electric double layer is generated and the anion toroidal vortex is generated around the initiator sequence. The disclosure provides the reaction site includes an amorphous silicon substrate where electrical conductivity is increased by illumination of light. The disclosure provides the anion toroidal vortex is created by generation of an anion electric double layer and AC electroosmosis. The disclosure provides the reaction site includes an amorphous silicon substrate where electrical conductivity is increased by illumination of light reflected by a spatial light modulator. The disclosure provides the one or more reaction reagents are removed from the reaction site by a volume of wash fluid. The disclosure provides the one or more reaction reagents are delivered by microfluidics. The disclosure provides the selected nucleotide is a natural nucleotide or a nucleotide analog.

The disclosure provides for individually controlling enzymatic activity in each reaction region on a substrate to produce prearranged oligonucleotide sequences in parallel to provide a method for multiplex manufacture of a plurality of oligonucleotides or polynucleotides.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts in schematic the use of TdT to add a nucleotide to an oligonucleotide.

FIG. 2 depicts in schematic the use of an anion toroidal vortex to move cations away from oligonucleotides attached to a reaction site on a substrate.

FIG. 3 depicts an arrangement, system or device generating an anion toroidal vortex.

FIG. 4 depicts aspects of an optically addressable virtual electrode.

FIG. 5 depicts aspects of AC electroosmosis and a virtual electrode.

FIG. 6 depicts an arrangement, system or device generating an anion toroidal vortex.

### DETAILED DESCRIPTION

The present disclosure is directed to the oligonucleotide sequences or polynucleotide sequences, whether random or designed, that are synthesized using enzymatic oligonucleotide synthesis reactions where an enzyme and a nucleotide (and related reagents or conditions) are placed at a desired site on a substrate under appropriate reaction conditions and the nucleotide is covalently bound to an existing nucleotide, such as an initiator sequence, which may be attached to a support. The oligonucleotide sequences may be synthesized using polymerases, such as error-prone polymerases under conditions where the reagents are localized at a location on a substrate for a period of time and under such conditions to maximize probability of adding a single nucleotide or desired number of nucleotides. The present disclosure provides that the enzyme is deactivated after the desired number of nucleotides have been covalently added. A suitable wash may also be used at a desired time to remove one or more reagents from the reaction site or location. The reagents or wash may be added to a location or reaction site using any suitable fluidics system or other systems known to those of skill in then art.

Polymerases, including without limitation error-prone template-dependent polymerases, modified or otherwise, can be used to create nucleotide polymers having a random or known or desired sequence of nucleotides. Template-independent polymerases, whether modified or otherwise, can be used to create the nucleic acids de novo. Ordinary nucleotides are used, such as A, T/U, C or G. Nucleotides may be used which lack chain terminating moieties. Chain terminating nucleotides may not be used in the methods of making the nucleotide polymers. A template independent polymerase may be used to make the nucleic acid sequence. Such template independent polymerase may be error-prone which may lead to the addition of more than one nucleotide resulting in a homopolymer. Sensors, such as light activated sensors, metabolic products or chemicals, that are activated by ligands can be used with such polymerases.

Oligonucleotide sequences or polynucleotide sequences are synthesized using an error prone polymerase, such as template independent error prone polymerase, and common or natural nucleic acids, which may be unmodified. Initiator sequences or primers are attached to a substrate, such as a silicon dioxide substrate, at various locations whether known, such as in an addressable array, or random. Reagents including at least a selected nucleotide, a template independent polymerase and other reagents required for enzymatic activity of the polymerase are applied at one or more locations of the substrate where the initiator sequences are located and under conditions where the polymerase adds one or more than one or a plurality of the nucleotide to the initiator sequence to extend the initiator sequence. The nucleotides (“dNTPs”) may be applied or flow in periodic applications. Blocking groups or reversible terminators are not used with the dNTPs because the reaction conditions may be selected to be sufficient to limit or reduce the probability of enzymatic addition of the dNTP to one dNTP, i.e. one dNTP is added using the selected reaction conditions taking into consideration the reaction kinetics. Although, it is to be understood that nucleotides with blocking groups or reversible terminators can be used in certain embodiments. Nucleotides with blocking groups or reversible terminators are known to those of skill in the art. According to an additional embodiment when reaction conditions permit, more than one dNTP may be added to form a homopolymer run when common or natural nucleotides are used with a template independent error prone polymerase.

Polymerase activity may be modified using photo-chemical or electrochemical modulation as a reaction condition so as to minimize addition of dNTP beyond a single dNTP. A wash is then applied to the one or more locations to remove the reagents. The steps of applying the reagents and the wash are repeated until desired nucleic acids are created. According to one aspect, the reagents may be added to one or more than one or a plurality of locations on the substrate in series or in parallel or the reagents may contact the entire surface of the support, such as by flowing the reagents across the surface of the support. According to one aspect, the reaction conditions are determined, for example based on reaction kinetics or the activity of the polymerase, so as to limit the ability of the polymerase to attach more than one nucleotide to the end of the initiator sequence or the growing oligonucleotide.

In addition, according to certain embodiments, polymerases can be modulated to be light sensitive for light based methods. According to this aspect, light is modulated to tune the polymerase to add only a single nucleotide. The light is shone on individual locations or pixels of the substrate where the polymerase, the nucleotide and appropriate reagents and reaction conditions are present. In this manner, a nucleotide is added to an initiator sequence or an existing nucleotide as the polymerase is activated by the light.

A flow cell or other channel, such a microfluidic channel or microfluidic channels having an input and an output is used to deliver fluids including reagents, such as a polymerase, a nucleotide and other appropriate reagents and washes to particular locations on a substrate within the flow cell, such as within a reaction chamber. According to certain aspects, an anion toroidal vortex is activated and deactivated to selectively deactivate and activate locations on the substrate. In this manner, a desired location, such as a grid point on a substrate or array, can be provided with reaction conditions to facilitate covalent binding of a nucleotide to an initiator sequence, an existing nucleotide or an existing oligonucleotide and the reaction conditions can be provided, such as by activation of an anion toroidal vortex at the reactive site to prevent further attachment of an additional nucleotide at the same location. Then, reaction conditions to facilitate covalent binding of a nucleotide to an existing nucleotide can be provided to the same location in a method of making an oligonucleotide at that desired location. One of skill will recognize that reaction conditions will be based on dimensions of the substrate reaction region, reagents, concentrations, reaction temperature, and the structures used to create and deliver the reagents and washes. According to certain aspects, pH and other reactants and reaction conditions can be optimized for the use of TdT to add a dNTP to an existing nucleotide or oligonucleotide in a template independent manner. For example, Ashley et al., Virology 77, 367-375 (1977) hereby incorporated by reference in its entirety identifies certain reagents and reaction conditions for dNTP addition, such as initiator size, divalent cation and pH. TdT was reported to be active over a wide pH range with an optimal pH of 6.85. Methods of providing or delivering dNTP, rNTP or rNDP are useful in making nucleic acids. Release of a lipase or other membrane-lytic enzyme from pH-sensitive viral particles inside dNTP filled-liposomes is described in J Clin Microbiol. May 1988; 26(5): 804-807. Photo-caged rNTPs or dNTPs from which NTPs can be released, typically nitrobenzyl derivatives sensitive to 350 nm light, are commercially available from Lifetechnologies. Rhoposin or bacterio-opsin triggered signal transduction resulting in vesicular or other secretion of nucleotides is known in the art. With these methods for delivering dNTPs, the nucleotides should be removed or sequestered between the first primer-polymerase encountered and any downstream.

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

### Nucleic Acids and Nucleotides

As used herein, the terms “nucleic acid molecule,” “nucleic acid sequence,” “nucleic acid fragment” and “oligomer” are used interchangeably and are intended to include, but are not limited to, a polymeric form of nucleotides that may have various lengths, including either deoxyribonucleotides or ribonucleotides, or analogs thereof.

In general, the terms “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs thereof. A oligonucleotide is typically composed of a specific sequence of four nucleotide bases: adenine (A); cytosine (C); guanine (G); and thymine (T) (uracil (U) for thymine (T) when the polynucleotide is RNA). According to certain aspects, deoxynucleotides (dNTPs, such as dATP, dCTP, dGTP, dTTP) may be used. According to certain aspects, ribonucleotide triphosphates (rNTPs) may be used. According to certain aspects, ribonucleotide diphosphates (rNDPs) may be used.

The term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides. The present disclosure contemplates any deoxyribonucleotide or ribonucleotide and chemical variants thereof, such as methylated, hydroxymethylated or glycosylated forms of the bases, and the like. According to certain aspects, natural nucleotides are used in the methods of making the nucleic acids. Natural nucleotides lack chain terminating moieties. According to another aspect, the methods of making the nucleic acids described herein do not use terminating nucleic acids or otherwise lack terminating nucleic acids, such as reversible terminators known to those of skill in the art. The methods are performed in the absence of chain terminating nucleic acids or wherein the nucleic acids are other than chain terminating nucleic acids.

Examples of modified nucleotides include, but are not limited to diaminopurine, S2T, 5-fluorouracil, 5-bromouracil, 5-chlorouracil, 5-iodouracil, hypoxanthine, xantine, 4-acetylcyto sine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A (2012) KlenTaq polymerase replicates unnatural base pairs by inducing a Watson-Crick geometry, Nature Chem. Biol. 8:612-614; See Y J, Malyshev D A, Lavergne T, Ordoukhanian P, Romesberg F E. J Am Chem Soc. 2011 Dec. 14; 133(49):19878-88, Site-specific labeling of DNA and RNA using an efficiently replicated and transcribed class of unnatural base pairs; Switzer C Y, Moroney S E, Benner S A. (1993) Biochemistry. 32(39):10489-96. Enzymatic recognition of the base pair between isocytidine and isoguanosine; Yamashige R, Kimoto M, Takezawa Y, Sato A, Mitsui T, Yokoyama S, Hirao I. Nucleic Acids Res. 2012 March; 40(6):2793-806. Highly specific unnatural base pair systems as a third base pair for PCR amplification; and Yang Z, Chen F, Alvarado J B, Benner S A. J Am Chem Soc. 2011 Sep. 28; 133(38):15105-12, Amplification, mutation, and sequencing of a six-letter synthetic genetic system. Other non-standard nucleotides may be used such as dexfribed in Malyshev, D. A., et al., Nature, vol. 509, pp. 385-388 (15 May 2014) hereby incorporated by reference in its entirety.

### Polymerases

According to an alternate embodiment of the present invention, polymerases are used to build nucleic acid molecules representing information which is referred to herein as being recorded in the nucleic acid sequence or the nucleic acid is referred to herein as being storage media. Polymerases are enzymes that produce a nucleic acid sequence, for example, using DNA or RNA as a template. Polymerases that produce RNA polymers are known as RNA polymerases, while polymerases that produce DNA polymers are known as DNA polymerases. Polymerases that incorporate errors are known in the art and are referred to herein as an “error-prone polymerases”. Template independent polymerases may be error prone polymerases. Using an error-prone polymerase allows the incorporation of specific bases at precise locations of the DNA molecule. Error-prone polymerases will either accept a non-standard base, such as a reversible chain terminating base, or will incorporate a different nucleotide, such as a natural or unmodified nucleotide that is selectively given to it as it tries to copy a template. Template-independent polymerases such as terminal deoxynucleotidyl transferase (TdT), also known as DNA nucleotidylexotransferase (DNTT) or terminal transferase create nucleic acid strands by catalyzing the addition of nucleotides to the 3′ terminus of a DNA molecule without a template. The preferred substrate of TdT is a 3′-overhang, but it can also add nucleotides to blunt or recessed 3′ ends. Cobalt is a cofactor, however the enzyme catalyzes reaction upon Mg and Mn administration in vitro. Nucleic acid initiators may be 4 or 5 nucleotides or longer and may be single stranded or double stranded. Double stranded initiators may have a 3′ overhang or they may be blunt ended or they may have a 3′ recessed end.

TdT, like all DNA polymerases, also requires divalent metal ions for catalysis. However, TdT is unique in its ability to use a variety of divalent cations such as Co2+, Mn2+, Zn2+ and Mg2+. In general, the extension rate of the primer p(dA)n (where n is the chain length from 4 through 50) with dATP in the presence of divalent metal ions is ranked in the following order: Mg2+>Zn2+>Co2+>Mn2+. In addition, each metal ion has different effects on the kinetics of nucleotide incorporation. For example, Mg2+ facilitates the preferential utilization of dGTP and dATP whereas Co2+ increases the catalytic polymerization efficiency of the pyrimidines, dCTP and dTTP. Zn2+ behaves as a unique positive effector for TdT since reaction rates with Mg2+ are stimulated by the addition of micromolar quantities of Zn2+. This enhancement may reflect the ability of Zn2+ to induce conformational changes in TdT that yields higher catalytic efficiencies. Polymerization rates are lower in the presence of Mn2+ compared to Mg2+, suggesting that Mn2+ does not support the reaction as efficiently as Mg2+. Further description of TdT is provided in Biochim Biophys Acta., May 2010; 1804(5): 1151-1166 hereby incorporated by reference in its entirety. In addition, one may replace Mg2+, Zn2+, Co2+, or Mn2+ in the nucleotide pulse with other cations designed modulate nucleotide attachment. For example, if the nucleotide pulse replaces Mg++ with other cation(s), such as Na+, K+, Rb+, Be++, Ca++, or Sr++, then the nucleotide can bind but not incorporate, thereby regulating whether the nucleotide will incorporate or not. Then a pulse of (optional) pre-wash without nucleotide or Mg++ can be provided or then Mg++ buffer without nucleotide can be provided.

By limiting nucleotides available to the polymerase, the incorporation of specific nucleic acids into the polymer can be regulated. Thus, these polymerases are capable of incorporating nucleotides independent of the template sequence and are therefore beneficial for creating nucleic acid sequences de novo. The combination of an error-prone polymerase and a primer sequence serves as a writing mechanism for imparting information into a nucleic acid sequence.

By limiting nucleotides available to a template independent polymerase, the addition of a nucleotide to an initiator sequence or an existing nucleotide or oligonucleotide can be regulated to produce an oligonucleotide by extension. Thus, these polymerases are capable of incorporating nucleotides without a template sequence and are therefore beneficial for creating nucleic acid sequences de novo.

The eta-polymerase (Matsuda et al. (2000) Nature 404(6781):1011-1013) is an example of a polymerase having a high mutation rate (˜10%) and high tolerance for 3′ mismatch in the presence of all 4 dNTPs and probably even higher if limited to one or two dNTPs. Hence, the eta-polymerase is a de novo recorder of nucleic acid information similar to terminal deoxynucleotidyl transferase (TdT) but with the advantage that the product produced by this polymerase is continuously double-stranded. Double stranded DNA has less sticky secondary structure and has a more predictable secondary structure than single stranded DNA. Furthermore, double stranded DNA serves as a good support for polymerases and/or DNA-binding-protein tethers.

According to certain aspects, a template dependent or template semi-dependent error prone polymerase can be used. According to certain embodiments, a template dependent polymerase may be used which may become error prone. According to certain embodiments, a template independent RNA polymerase can be used. Where a template dependent or template semi-dependent polymerase is used, any combination of templates with universal bases can be used which encourage acceptance of many nucleotide types. In addition, error tolerant cations such as Mn⁺ can be used. Further, the present disclosure contemplates the use of error-tolerant polymerase mutants. See Berger et al., Universal Bases for Hybridization, Replication and Chain Termination, Nucleic Acids Research 2000, August 1, 28(15) pp. 2911-2914 hereby incorporated by reference.

Nucleic acids that have been synthesized on the surface of a support may be removed, such as by a cleavable linker or linkers known to those of skill in the art. The nucleic acids may be positioned on a different substrate, such as at a higher density than the manufacturing density, or on a different substrate that is to serve as the storage medium. Also, additional layers of substrates may be added which serve as new substrates for additional nucleic acid synthesis. Accordingly, methods are provided to make a high density nucleic acid storage device by generating a plurality of oligonucleotides on a first substrate, removing the plurality of oligonucleotides from the first substrate and attaching them to a second substrate in a random or ordered manner and with a desired density.

### Supports and Attachment

In certain exemplary embodiments, one or more oligonucleotide sequences described herein are immobilized on a support (e.g., a solid and/or semi-solid support). In certain aspects, an oligonucleotide sequence can be attached to a support using one or more of the phosphoramidite linkers described herein. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. Supports of the present invention can be any shape, size, or geometry as desired. For example, the support may be square, rectangular, round, flat, planar, circular, tubular, spherical, and the like. When using a support that is substantially planar, the support may be physically separated into regions, for example, with trenches, grooves, wells, or chemical barriers (e.g., hydrophobic coatings, etc.). Supports may be made from glass (silicon dioxide), metal, ceramic, polymer or other materials known to those of skill in the art. Supports may be a solid, semi-solid, elastomer or gel. In certain exemplary embodiments, a support is a microarray. As used herein, the term “microarray” refers in one embodiment to a type of array that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate create a substantially planar surface. Spatially defined sites may additionally be “addressable” in that its location and the identity of the immobilized probe at that location are known or determinable.

The solid supports can also include a semi-solid support such as a compressible matrix with both a solid and a liquid component, wherein the liquid occupies pores, spaces or other interstices between the solid matrix elements. Preferably, the semi-solid support materials include polyacrylamide, cellulose, poly dimethyl siloxane, polyamide (nylon) and cross-linked agarose, -dextran and -polyethylene glycol. Solid supports and semi-solid supports can be used together or independent of each other.

Supports can also include immobilizing media. Such immobilizing media that are of use according to the invention are physically stable and chemically inert under the conditions required for nucleic acid molecule deposition and amplification. A useful support matrix withstands the rapid changes in, and extremes of, temperature required for PCR. The support material permits enzymatic nucleic acid synthesis. If it is unknown whether a given substance will do so, it is tested empirically prior to any attempt at production of a set of arrays according to the invention. According to one embodiment of the present invention, the support structure comprises a semi-solid (i.e., gelatinous) lattice or matrix, wherein the interstices or pores between lattice or matrix elements are filled with an aqueous or other liquid medium; typical pore (or ‘sieve’) sizes are in the range of 100 μm to 5 nm. Larger spaces between matrix elements are within tolerance limits, but the potential for diffusion of amplified products prior to their immobilization is increased. The semi-solid support is compressible. The support is prepared such that it is planar, or effectively so, for the purposes of printing. For example, an effectively planar support might be cylindrical, such that the nucleic acids of the array are distributed over its outer surface in order to contact other supports, which are either planar or cylindrical, by rolling one over the other. Lastly, a support material of use according to the invention permits immobilizing (covalent linking) of nucleic acid features of an array to it by means known to those skilled in the art. Materials that satisfy these requirements comprise both organic and inorganic substances, and include, but are not limited to, polyacrylamide, cellulose and polyamide (nylon), as well as cross-linked agarose, dextran or polyethylene glycol.

One embodiment is directed to a thin polyacrylamide gel on a glass support, such as a plate, slide or chip. A polyacrylamide sheet of this type is synthesized as follows. Acrylamide and bis-acrylamide are mixed in a ratio that is designed to yield the degree of crosslinking between individual polymer strands (for example, a ratio of 38:2 is typical of sequencing gels) that results in the desired pore size when the overall percentage of the mixture used in the gel is adjusted to give the polyacrylamide sheet its required tensile properties. Polyacrylamide gel casting methods are well known in the art (see Sambrook et al., 1989, Molecular Cloning. A Laboratory Manual, 2nd Edition, Cold Spring Harbor Laboratory Press, Cold Spring Harbor, N.Y., incorporated herein in its entirety by reference), and one of skill has no difficulty in making such adjustments.

The gel sheet is cast between two rigid surfaces, at least one of which is the glass to which it will remain attached after removal of the other. The casting surface that is to be removed after polymerization is complete is coated with a lubricant that will not inhibit gel polymerization; for this purpose, silane is commonly employed. A layer of silane is spread upon the surface under a fume hood and allowed to stand until nearly dry. Excess silane is then removed (wiped or, in the case of small objects, rinsed extensively) with ethanol. The glass surface which will remain in association with the gel sheet is treated with γ-methacryloxypropyltrimethoxysilane (Cat. No. M6514, Sigma; St. Louis, Mo.), often referred to as ‘crosslink silane’, prior to casting. The glass surface that will contact the gel is triply-coated with this agent. Each treatment of an area equal to 1200 cm² requires 125 μl of crosslink silane in 25 ml of ethanol Immediately before this solution is spread over the glass surface, it is combined with a mixture of 750 μl water and 75 μl glacial acetic acid and shaken vigorously. The ethanol solvent is allowed to evaporate between coatings (about 5 minutes under a fume hood) and, after the last coat has dried, excess crosslink silane is removed as completely as possible via extensive ethanol washes in order to prevent ‘sandwiching’ of the other support plate onto the gel. The plates are then assembled and the gel cast as desired.

The only operative constraint that determines the size of a gel that is of use according to the invention is the physical ability of one of skill in the art to cast such a gel. The casting of gels of up to one meter in length is, while cumbersome, a procedure well known to workers skilled in nucleic acid sequencing technology. A larger gel, if produced, is also of use according to the invention. An extremely small gel is cut from a larger whole after polymerization is complete.

Note that at least one procedure for casting a polyacrylamide gel with bioactive substances, such as enzymes, entrapped within its matrix is known in the art (O'Driscoll, 1976, Methods Enzymol., 44: 169-183, incorporated herein in its entirety by reference). A similar protocol, using photo-crosslinkable polyethylene glycol resins, that permit entrapment of living cells in a gel matrix has also been documented (Nojima and Yamada, 1987, Methods Enzymol., 136: 380-394, incorporated herein in its entirety by reference). Such methods are of use according to the invention. As mentioned below, whole cells are typically cast into agarose for the purpose of delivering intact chromosomal DNA into a matrix suitable for pulsed-field gel electrophoresis or to serve as a “lawn” of host cells that will support bacteriophage growth prior to the lifting of plaques according to the method of Benton and Davis (see Maniatis et al., 1982, Molecular Cloning: A Laboratory Manual, Cold Spring Harbor Laboratory Press, Cold Spring Harbor, N.Y., incorporated herein in its entirety by reference). In short, electrophoresis-grade agarose (e.g., Ultrapure; Life Technologies/Gibco-BRL) is dissolved in a physiological (isotonic) buffer and allowed to equilibrate to a temperature of 50° C. to 52° C. in a tube, bottle or flask. Cells are then added to the agarose and mixed thoroughly, but rapidly (if in a bottle or tube, by capping and inversion, if in a flask, by swirling), before the mixture is decanted or pipetted into a gel tray. If low-melting point agarose is used, it may be brought to a much lower temperature (down to approximately room temperature, depending upon the concentration of the agarose) prior to the addition of cells. This is desirable for some cell types; however, if electrophoresis is to follow cell lysis prior to covalent attachment of the molecules of the resultant nucleic acid pool to the support, it is performed under refrigeration, such as in a 4° C. to 10° C. ‘cold’ room.

Oligonucleotides immobilized on microarrays include nucleic acids that are generated in or from an assay reaction. Typically, the oligonucleotides or polynucleotides on microarrays are single stranded and are covalently attached to the solid phase support, usually by a 5′-end or a 3′-end. In certain exemplary embodiments, probes are immobilized via one or more cleavable linkers. The density of non-overlapping regions containing nucleic acids in a microarray is typically greater than 100 per cm², and more typically, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al, U.S. Pat. Nos. 5,424,186; 5,445,934; and 5,744,305.

Methods of immobilizing oligonucleotides to a support are known in the art (beads: Dressman et al. (2003) Proc. Natl. Acad. Sci. USA 100:8817, Brenner et al. (2000) Nat. Biotech. 18:630, Albretsen et al. (1990) Anal. Biochem. 189:40, and Lang et al. Nucleic Acids Res. (1988) 16:10861; nitrocellulose: Ranki et al. (1983) Gene 21:77; cellulose: Goldkorn (1986) Nucleic Acids Res. 14:9171; polystyrene: Ruth et al. (1987) Conference of Therapeutic and Diagnostic Applications of Synthetic Nucleic Acids, Cambridge U.K.; teflon-acrylamide: Duncan et al. (1988) Anal. Biochem. 169:104; polypropylene: Polsky-Cynkin et al. (1985) Clin. Chem. 31:1438; nylon: Van Ness et al. (1991) Nucleic Acids Res. 19:3345; agarose: Polsky-Cynkin et al., Clin. Chem. (1985) 31:1438; and sephacryl: Langdale et al. (1985) Gene 36:201; latex: Wolf et al. (1987) Nucleic Acids Res. 15:2911). Supports may be coated with attachment chemistry or polymers, such as amino-silane, NHS-esters, click chemistry, polylysine, etc., to bind a nucleic acid to the support.

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

According to certain aspects, affixing or immobilizing nucleic acid molecules to the substrate is performed using a covalent linker that is selected from the group that includes oxidized 3-methyl uridine, an acrylyl group and hexaethylene glycol. In addition to the attachment of linker sequences to the molecules of the pool for use in directional attachment to the support, a restriction site or regulatory element (such as a promoter element, cap site or translational termination signal), is, if desired, joined with the members of the pool. Linkers can also be designed with chemically reactive segments which are optionally cleavable with agents such as enzymes, light, heat, pH buffers, and redox reagents. Such linkers can be employed to pre-fabricate an in situ solid-phase inactive reservoir of a different solution-phase primer for each discrete feature. Upon linker cleavage, the primer would be released into solution for PCR, perhaps by using the heat from the thermocycling process as the trigger.

It is also contemplated that affixing of nucleic acid molecules to the support is performed via hybridization of the members of the pool to nucleic acid molecules that are covalently bound to the support.

Immobilization of nucleic acid molecules to the support matrix according to the invention is accomplished by any of several procedures. Direct immobilizing via the use of 3′-terminal tags bearing chemical groups suitable for covalent linkage to the support, hybridization of single-stranded molecules of the pool of nucleic acid molecules to oligonucleotide primers already bound to the support, or the spreading of the nucleic acid molecules on the support accompanied by the introduction of primers, added either before or after plating, that may be covalently linked to the support, may be performed. Where pre-immobilized primers are used, they are designed to capture a broad spectrum of sequence motifs (for example, all possible multimers of a given chain length, e.g., hexamers), nucleic acids with homology to a specific sequence or nucleic acids containing variations on a particular sequence motif. Alternatively, the primers encompass a synthetic molecular feature common to all members of the pool of nucleic acid molecules, such as a linker sequence.

Two means of crosslinking a nucleic acid molecule to a polyacrylamide gel sheet will be discussed in some detail. The first (provided by Khrapko et al., 1996, U.S. Pat. No. 5,552,270) involves the 3′ capping of nucleic acid molecules with 3-methyl uridine. Using this method, the nucleic acid molecules of the libraries of the present invention are prepared so as to include this modified base at their 3′ ends. In the cited protocol, an 8% polyacrylamide gel (30:1, acrylamide:bis-acrylamide) sheet 30 μm in thickness is cast and then exposed to 50% hydrazine at room temperature for 1 hour. Such a gel is also of use according to the present invention. The matrix is then air dried to the extent that it will absorb a solution containing nucleic acid molecules, as described below. Nucleic acid molecules containing 3-methyl uridine at their 3′ ends are oxidized with 1 mM sodium periodate (NaIO₄) for 10 minutes to 1 hour at room temperature, precipitated with 8 to 10 volumes of 2% LiClO₄ in acetone and dissolved in water at a concentration of 10 pmol/μl. This concentration is adjusted so that when the nucleic acid molecules are spread upon the support in a volume that covers its surface evenly and is efficiently (i.e., completely) absorbed by it, the density of nucleic acid molecules of the array falls within the range discussed above. The nucleic acid molecules are spread over the gel surface and the plates are placed in a humidified chamber for 4 hours. They are then dried for 0.5 hour at room temperature and washed in a buffer that is appropriate to their subsequent use. Alternatively, the gels are rinsed in water, re-dried and stored at −20° C. until needed. It is thought that the overall yield of nucleic acid that is bound to the gel is 80% and that of these molecules, 98% are specifically linked through their oxidized 3′ groups.

A second crosslinking moiety that is of use in attaching nucleic acid molecules covalently to a polyacrylamide sheet is a 5′ acrylyl group, which is attached to the primers. Oligonucleotide primers bearing such a modified base at their 5′ ends may be used according to the invention. In particular, such oligonucleotides are cast directly into the gel, such that the acrylyl group becomes an integral, covalently bonded part of the polymerizing matrix. The 3′ end of the primer remains unbound, so that it is free to interact with, and hybridize to, a nucleic acid molecule of the pool and prime its enzymatic second-strand synthesis.

Alternatively, hexaethylene glycol is used to covalently link nucleic acid molecules to nylon or other support matrices (Adams and Kron, 1994, U.S. Pat. No. 5,641,658). In addition, nucleic acid molecules are crosslinked to nylon via irradiation with ultraviolet light. While the length of time for which a support is irradiated as well as the optimal distance from the ultraviolet source is calibrated with each instrument used due to variations in wavelength and transmission strength, at least one irradiation device designed specifically for crosslinking of nucleic acid molecules to hybridization membranes is commercially available (Stratalinker, Stratagene). It should be noted that in the process of crosslinking via irradiation, limited nicking of nucleic acid strands occurs. The amount of nicking is generally negligible, however, under conditions such as those used in hybridization procedures. In some instances, however, the method of ultraviolet crosslinking of nucleic acid molecules will be unsuitable due to nicking. Attachment of nucleic acid molecules to the support at positions that are neither 5′- nor 3′-terminal also occurs, but it should be noted that the potential for utility of an array so crosslinked is largely uncompromised, as such crosslinking does not inhibit hybridization of oligonucleotide primers to the immobilized molecule where it is bonded to the support.

Supports described herein may have one or more optically addressable virtual electrodes associated therewith such that an anion toroidal vortex can be created at a reaction site on the supports described herein.

### Reagent Delivery Systems

According to certain aspects, reagents and washes are delivered that the reactants are present at a desired location for a desired period of time to, for example, covalently attached dNTP to an initiator sequence or an existing nucleotide attached at the desired location. A selected nucleotide reagent liquid is pulsed or flowed or deposited at the reaction site where reaction takes place and then may be optionally followed by delivery of a buffer or wash that does not include the nucleotide. Suitable delivery systems include fluidics systems, microfluidics systems, syringe systems, ink jet systems, pipette systems and other fluid delivery systems known to those of skill in the art. Various flow cell embodiments or flow channel embodiments or microfluidic channel embodiments are envisioned which can deliver separate reagents or a mixture of reagents or washes using pumps or electrodes or other methods known to those of skill in the art of moving fluids through channels or microfluidic channels through one or more channels to a reaction region or vessel where the surface of the substrate is positioned so that the reagents can contact the desired location where a nucleotide is to be added.

According to another embodiment, a microfluidic device is provided with one or more reservoirs which include one or more reagents which are then transferred via microchannels to a reaction zone where the reagents are mixed and the reaction occurs. Such microfluidic devices and the methods of moving fluid reagents through such microfluidic devices are known to those of skill in the art.

Immobilized nucleic acid molecules may, if desired, be produced using a device (e.g., any commercially-available inkjet printer, which may be used in substantially unmodified form) which sprays a focused burst of reagent-containing solution onto a support (see Castellino (1997) Genome Res. 7:943-976, incorporated herein in its entirety by reference). Such a method is currently in practice at Incyte Pharmaceuticals and Rosetta Biosystems, Inc., the latter of which employs “minimally modified Epson inkjet cartridges” (Epson America, Inc.; Torrance, Calif.). The method of inkjet deposition depends upon the piezoelectric effect, whereby a narrow tube containing a liquid of interest (in this case, oligonucleotide synthesis reagents) is encircled by an adapter. An electric charge sent across the adapter causes the adapter to expand at a different rate than the tube, and forces a small drop of liquid reagents from the tube onto a coated slide or other support.

Reagents can be deposited onto a discrete region of the support, such that each region forms a feature of the array. The feature is capable of generating an anion toroidal vortex as described herein. The desired nucleic acid sequence can be synthesized drop-by-drop at each position, as is true for other methods known in the art. If the angle of dispersion of reagents is narrow, it is possible to create an array comprising many features. Alternatively, if the spraying device is more broadly focused, such that it disperses nucleic acid synthesis reagents in a wider angle, as much as an entire support is covered each time, and an array is produced in which each member has the same sequence (i.e., the array has only a single feature).

There are contemplated different distributions for the time for binding a nucleotide precursor (dNTP/rNTP/rNDP) and time spent in making the covalent bond with the growing primer 3′ end. An array-based, flow-cell technique is used, similar to standard synthesis and sequencing procedures. Starting TdT primers are bonded to flat silicon dioxide (or 10 micron thick polymer layer) at known locations which are capable of generating an anion toroidal vortex as described herein. Locations for creating oligonucleotides can range in number between 1,000 and 5,000,000.

### Example I

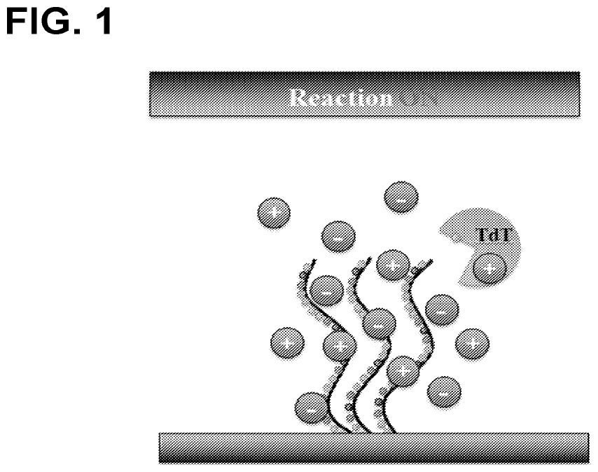

FIG. 1 depicts in schematic single stranded initiator nucleic acid sequences (or growing oligonucleotide sequences) attached to a substrate. The substrate is designed as described herein to generate and anion toroidal vortex. When the anion toroidal vortex is not generated, local anions and cations and TdT are capable of interacting with the initiator nucleic acid sequences to facilitate addition of a nucleotide to the initiator sequence. The present disclosure contemplates individually controlling the enzymatic activity in each reaction spot to produce prearranged oligonucleotide sequences in parallel. Moving local ion concentration away from the initiator sequence effectively deactivates the enzyme from adding a nucleotide to the initiator.

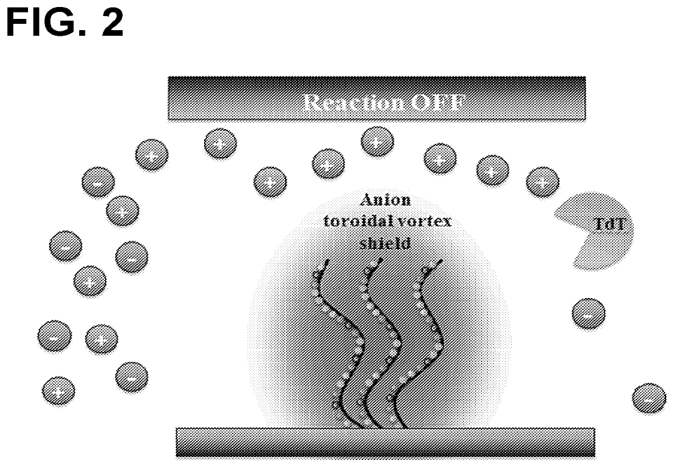

FIG. 2 depicts in schematic the generation of an anion toroidal vortex around the single stranded initiator nucleic acid sequences (or growing oligonucleotide sequences which may be probes or other useful nucleic acid sequence) attached to a substrate. Generation of the anion toroidal vortex forces or moves the ions away from the single stranded initiator nucleic acid sequences making them unavailable for use by the enzyme to add a nucleotide to the single stranded initiator nucleic acid sequences. The forcing or moving away of the ions, such as cations and anions, from the single stranded initiator nucleic acid sequences minimizes the chances of nucleotide addition to the extent that the cations are needed for enzymatic addition of a nucleotide.

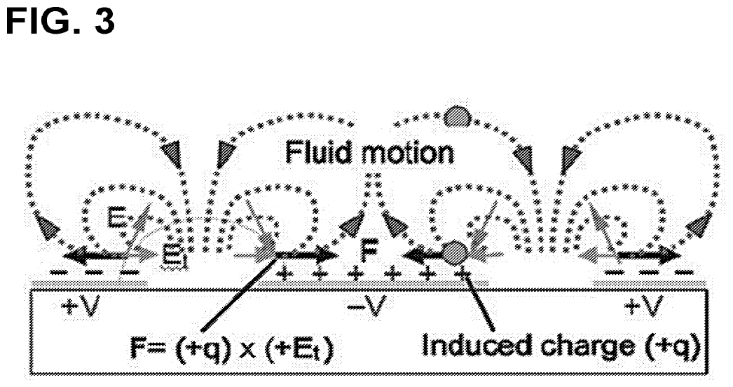

FIG. 3 depicts an arrangement or device which may be used to generate an anion toroidal vortex. The device includes or is configured to produce an electric double layer (EDL) and to provide for AC electroosmosis. FIG. 3 depicts electrokinetic motion of counter ions in the EDL under non-uniform electric potential.

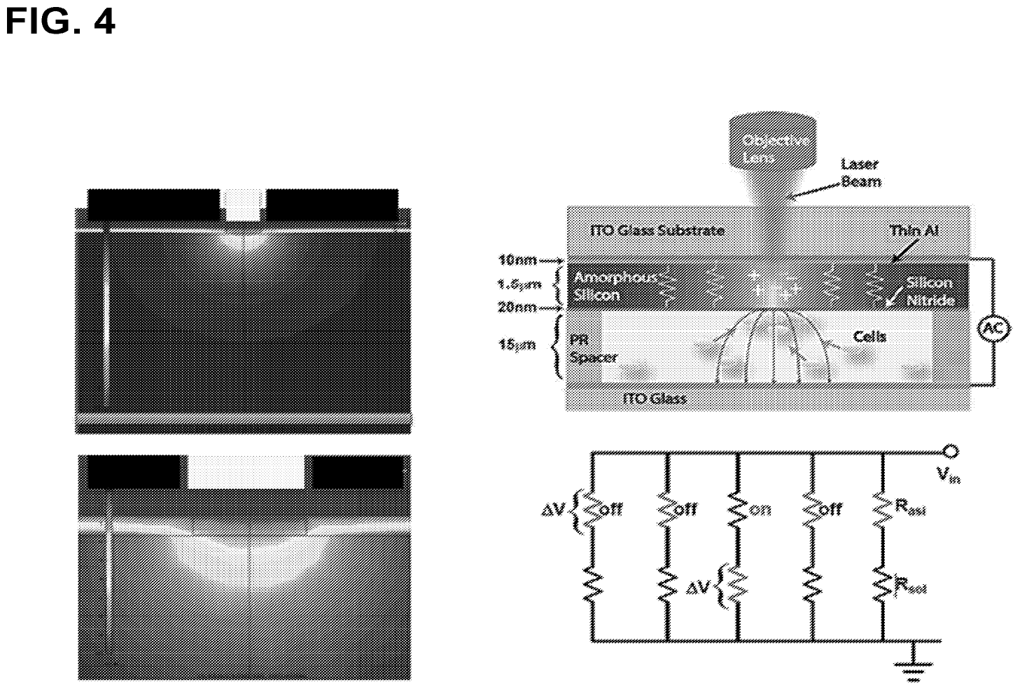

FIG. 4 depicts an optically addressable virtual electrode of the present disclosure which includes a light activated conductivity changing thin layer of amorphous silicon. A light source illuminates the light activated conductivity changing thin layer of amorphous silicon to alter the electric impedance thereby generating a tangential electric field. The optically addressable virtual electrode device generates local anion electric double layer and generating anion toroidal vortex around the ssDNA binding area. According to the intensity of light pattern, the electric impedance of the amorphous silicon layer changes, thereby generating a tangential electric field. Under the action of a tangential electric field, excess counterions (anions) experience a net electrostatic force then forms an anion toroidal vortex. Since the ssDNA probes in this active electrode region have minimum chance to meet cations, the activity of TdT enzyme is selectively controlled, thereby spatially controlling the incorporation of deoxynucleotide in a single microfluidics reaction chamber.

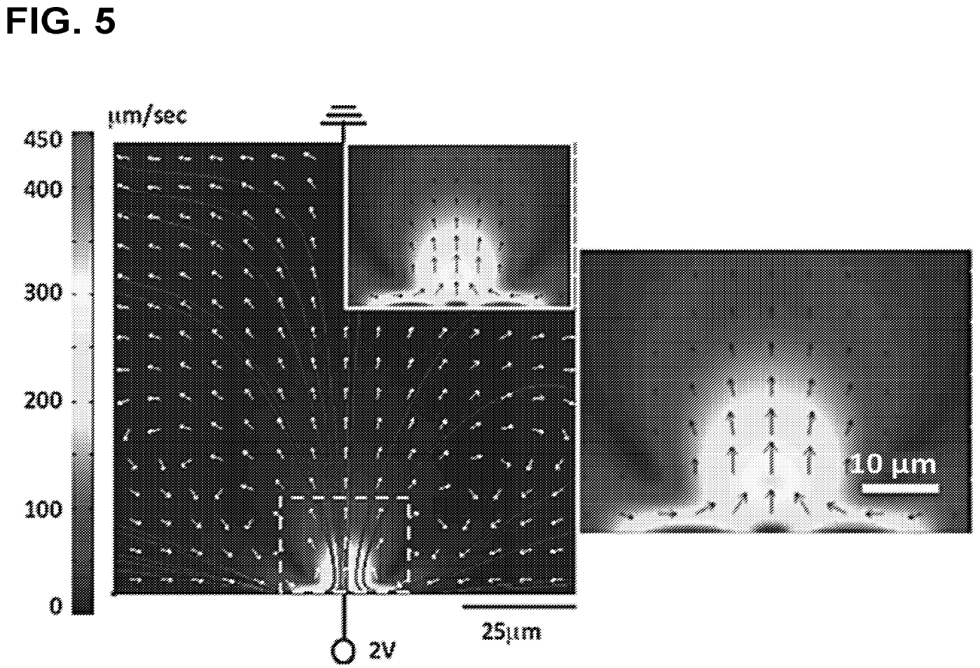

FIG. 5 depicts application of AC electroosmosis using a virtual electrode.

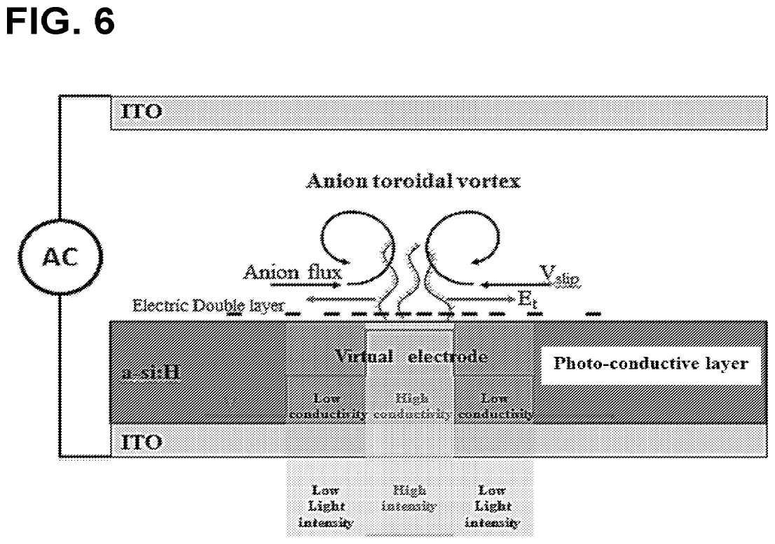

FIG. 6 depicts in schematic the device of the present disclosure that is used to generate the anion toroidal vortex at the reaction site on or associated with a substrate. A light intensity difference, creates a voltage difference which generates a tangential electric field which promote counter-ion movement which creates the anion toroidal vortex around the nucleic acids on the substrate. The amorphous silicon layer includes an area of high conductivity bounded by areas of low conductivity which create a virtual electrode within the photoconductive layer. FIG. 6 depicts the electric double layer, the anion flux and the anion toroidal vortex created at the reaction site of the substrate.

### OTHER EMBODIMENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

1. A method for making a polynucleotide comprising
(a) delivering one or more reaction reagents including an error prone or template independent DNA polymerase, cations and a selected nucleotide to a reaction site including an initiator sequence having a terminal nucleotide for a time period and under conditions sufficient to covalently add a desired number of the selected nucleotide to the terminal nucleotide at the 3′ end of the initiator such that the selected nucleotide becomes a terminal nucleotide,
moving cations away from the initiator sequence using an anion toroidal vortex to inhibit covalent addition of the selected nucleotide by the error prone or template independent DNA polymerase,
removing the reaction reagents from the reaction site, and
(b) repeating step (a) until the polynucleotide is formed.

2. The method of claim 1 wherein the anion toroidal vortex deactivates the error prone or template independent DNA polymerase.

3. The method of claim 1 wherein the anion toroidal vortex localizes the cations away from the initiator sequence.

4. The method of claim 1 wherein the anion toroidal vortex controls activity of the error prone or template independent DNA polymerase at the reaction site.

5. The method of claim 1 wherein a single selected nucleotide is covalently added.

6. The method of claim 1 wherein the error prone template independent DNA polymerase is terminal deoxynucleotide transferase.

7. The method of claim 1 wherein the anion toroidal vortex is created by an optically addressable virtual electrode.

8. The method of claim 1 including a plurality of reaction sites where step (a) is performed.

9. The method of claim 1 wherein the reaction site includes an amorphous silicon layer where electric impedance changes in response to light intensity and a tangential electric field is generated.

10. The method of claim 1 wherein an anion electric double layer is generated and the anion toroidal vortex is generated around the initiator sequence.

11. The method of claim 1 wherein the reaction site includes an amorphous silicon substrate where electrical conductivity is increased by illumination of light.

12. The method of claim 1 wherein the anion toroidal vortex is created by generation of an anion electric double layer and AC electroosmosis.

13. The method of claim 1 wherein the reaction site includes an amorphous silicon substrate where electrical conductivity is increased by illumination of light reflected by a spatial light modulator.

14. The method of claim 1 wherein the reaction reagents are removed from the reaction site by a volume of wash fluid.

15. The method of claim 1 wherein the one or more reaction reagents are delivered by microfluidics.

16. The method of claim 1 wherein the selected nucleotide is a natural nucleotide or a nucleotide analog.
